(19) United States
(12) Patent Application Publication
(10) Pub. No.: US 2020/0285804 A1
(43) Pub. Date: Sep. 10, 2020

(54) Systems and Methods for Generating Context-Aware Word Embeddings

(71) Applicant: The Board of Trustees of the Leland Stanford Junior University, Stanford, CA (US)

(72) Inventors: Daniel L. Rubin, Stanford, CA (US); Imon Banerjee, Stanford, CA (US)

(73) Assignee: The Board of Trustees of the Leland Stanford Junior University, Stanford, CA (US)

(21) Appl. No.: 16/810,719

(22) Filed: Mar. 5, 2020

Related U.S. Application Data
(60) Provisional application No. 62/814,225, filed on Mar. 5, 2019.

(51) Int. Cl. G06F 40/169; G06F 40/242; G16H 15/00; G06F 16/36; G06F 16/33

(52) U.S. Cl. CPC G06F 40/169; G06F 40/242; G06F 16/3347; G06F 16/367; G16H 15/00

(57) ABSTRACT

Systems and methods for generating context-aware word embeddings in accordance with embodiments of the invention are illustrated. One embodiment includes a report annotation server, including a processor; and a memory containing a report annotation application, where the report annotation application configures the processor to obtain a plurality of case reports from at least one medical database, preprocess the plurality of case reports, segment the preprocessed plurality of case reports, reduce the term ambiguity of the segmented plurality of case reports, generate word embeddings, and generate a context-aware vector based on the word embeddings.

### CROSS-REFERENCE TO RELATED APPLICATIONS

The current application claims the benefit of and priority under 35 U.S.C. § 119(e) to U.S. Provisional Patent Application No. 62/814,225 entitled “Automated Annotation of Text Reports to Enable Developing AI Applications” filed Mar. 5, 2019. The disclosure of U.S. Provisional Patent Application No. 62/814,225 is hereby incorporated by reference in its entirety for all purposes.

### FIELD OF THE INVENTION

The present invention generally relates to the automated creation of word embeddings from documents, and more specifically the annotation of radiology report databases to using context-aware word embeddings.

### BACKGROUND

Natural language processing (NLP) is a cross-disciplinary field of study concerned with the interactions between computers and natural human language. Word embedding is a subfield of NLP where words or phrases from a vocabulary are mapped to vectors of real numbers.

Radiology is a field of medicine concerned with diagnosing and treating injuries and diseases using medical imaging procedures. Example medical imaging procedures include, but are not limited to, X-rays, computed tomography (CT), magnetic resonance imaging (MRI), nuclear medicine, positron emission tomography (PET), and ultrasound. Medical imaging devices are useful as they enable observation of internal structures and tissues of a body without invasive procedures.

### SUMMARY OF THE INVENTION

Systems and methods for generating context-aware word embeddings in accordance with embodiments of the invention are illustrated. One embodiment includes a report annotation server, including a processor; and a memory containing a report annotation application, where the report annotation application configures the processor to obtain a plurality of case reports from at least one medical database, preprocess the plurality of case reports, segment the preprocessed plurality of case reports, reduce the term ambiguity of the segmented plurality of case reports, generate word embeddings, and generate a context-aware vector based on the word embeddings.

In another embodiment, the report annotation application further configures the processor to annotate case reports in the plurality of case reports based on the context-aware vectors.

In a further embodiment, case reports in the plurality of case reports comprise radiology images.

In still another embodiment, case reports in the plurality of case reports conform to the AIM file standard.

In a still further embodiment, the report annotation application further directs the processor to segment the preprocessed plurality of case reports based on report section.

In yet another embodiment, to reduce the term ambiguity, the report annotation application further directs the processor to generate a domain ontology, and identify words in segmented plurality of case reports that map to key-terms in the domain ontology.

In a yet further embodiment, the domain ontology is based on a query of the RadLex lexicon.

In another additional embodiment, the query of the RadLex lexicon is merged with a general terminology dictionary.

In a further additional embodiment, to generate word embeddings, the report annotation application directs the processor to use a word2vec model.

In another embodiment again, to generate context aware vectors, the report annotation directs the processor to identify a window of relevant words based on the location of an identified key-term.

In a further embodiment again, a method for annotation reports including obtaining a plurality of case reports from at least one medical database, preprocessing the plurality of case reports, segmenting the preprocessed plurality of case reports, reducing the term ambiguity of the segmented plurality of case reports, generating word embeddings, and generating a context-aware vector based on the word embeddings.

In still yet another embodiment, the method further includes annotating case reports in the plurality of case reports based on the context-aware vectors.

In a still yet further embodiment, case reports in the plurality of case reports comprise radiology images.

In still another additional embodiment, case reports in the plurality of case reports conform to the AIM file standard.

In a still further additional embodiment, segmenting the preprocessed plurality of case reports is based on report section.

In still another embodiment again, reducing term ambiguity includes generating a domain ontology, and identifying words in segmented plurality of case reports that map to key-terms in the domain ontology.

In a still further embodiment again, the domain ontology is based on a query of the RadLex lexicon.

In yet another additional embodiment, the query of the RadLex lexicon is merged with a general terminology dictionary.

In a yet further additional embodiment, generating word embeddings comprises using a word2vec model.

In yet another embodiment again, generating context aware vectors comprises identifying a window of relevant words based on the location of an identified key-term.

Additional embodiments and features are set forth in part in the description that follows, and in part will become apparent to those skilled in the art upon examination of the specification or may be learned by the practice of the invention. A further understanding of the nature and advantages of the present invention may be realized by reference to the remaining portions of the specification and the drawings, which forms a part of this disclosure.

### BRIEF DESCRIPTION OF THE DRAWINGS

The description and claims will be more fully understood with reference to the following figures, which are presented as exemplary embodiments of the invention and should not be construed as a complete recitation of the scope of the invention.

FIG. 1 illustrates a report annotation system in accordance with an embodiment of the invention.

FIG. 2 illustrates a report annotation server in accordance with an embodiment of the invention.

FIG. 3 is a flowchart for a report annotation process in accordance with an embodiment of the invention.

### DETAILED DESCRIPTION

Artificial intelligence (AI) technologies are developing rapidly, and there is an explosion in commercial activity in developing AI applications. Specifically, machine learning (ML) models have seen considerable success in fields such as image processing and NLP. However, many ML models, such as supervised learning models, require a training process using matched inputs and outputs, referred to as training data. In many fields where ML would be useful, there is an unfortunate dearth of training data. For example, in the radiology space, while there are a considerable number of raw case reports, they are often heterogeneous in form and it would be difficult to manually annotate the raw case reports such that they are usable as training data. Systems and methods described herein can remedy this problem by automating the collection of case reports and annotating them such that they can be used as training data for ML models.

In various embodiments, systems and methods described herein can obtain a corpus of raw case reports and generate a word embedding for each report that can be used to annotate the report. A number of different case report standards are in use, many of which are disease specific, such as, the Reporting and Data System (RADS) atlas standards (e.g. BI-RADS, LI-RADS, C-RADS, Lung-RADS, etc.). However, despite various standards, doctor's reports often contain natural language notes and/or labels that may be unique to a particular doctor or medical institution's vernacular. Consequently, it is difficult to utilize reports from multiple institutions as training data. By generating context-aware word embedding vectors, a heterogeneous set of reports can be transformed into a homogenous training data set. Systems for acquiring raw case reports and generating context-aware word embedding vectors are described in further detail below.

### Report Annotation Systems

Report annotation systems are capable of aggregating and transforming heterogenous raw case reports into a homogenous data set via annotation with context-aware word embedding vectors. In numerous embodiments, report annotation systems can generate training data sets for AI training applications. Report annotation systems can be architected in any number of ways, including, but not limited to, as a distributed system. A report annotation system in accordance with an embodiment of the invention is described below.

Report annotation system 100 includes a report annotation server 110. Report annotation server obtains raw case reports from medical institution (e.g. hospitals, clinics, etc.) servers 120 and medical database repositories 130 via a network 140. However, in numerous embodiments, report annotation servers obtain raw case reports from only one source. In various embodiments, the network is the Internet, a medical communications network, and/or any other wired and/or wireless network as appropriate to the requirements of specific applications of embodiments of the invention. In various embodiments, raw case reports can be directly provided via a physical storage media. Report annotation servers, medical institution servers, and medical database repositories can be implemented using one or more computing devices. Report annotation servers are discussed in further detail below.

### Report Annotation Servers

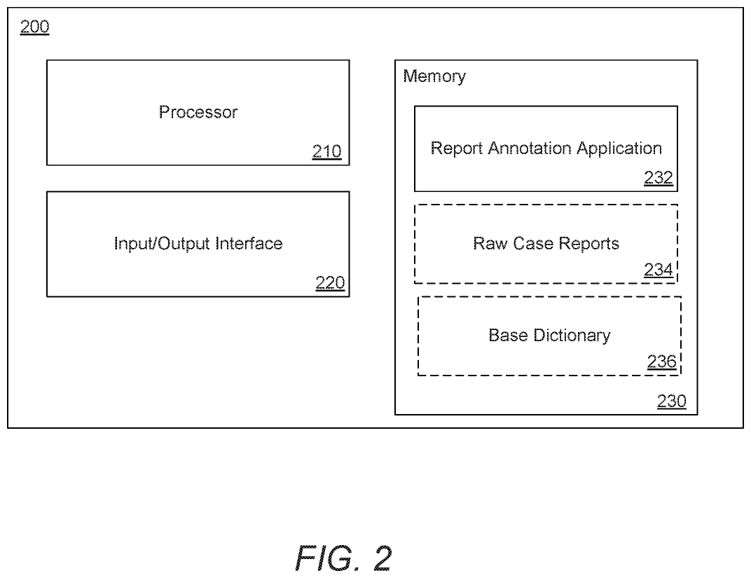

Report annotation servers are computing devices that can perform NLP processes to generate context-aware word embedding vectors. In numerous embodiments, report annotation servers can be integrated into AI training systems to as part of a training data generation system. A report annotation server in accordance with an embodiment of the invention is illustrated in FIG. 2.

Report annotation server 200 includes a processor 210. Processors are any circuit capable of performing logical calculations, including, but not limited to, central processing units (CPUs), graphics processing units (GPUs), field-programmable gate arrays (FPGAs), application-specific integrated circuits (ASICs), and/or any other circuit as appropriate to the requirements of specific applications of embodiments of the invention. Report annotation server 200 further incudes an input/output (I/O) interface (220). The I/O interface is capable of sending and receiving data to external devices, including, but not limited to, collection servers. The report annotation server also includes a memory 230. The memory can be implemented as volatile memory, nonvolatile memory, and/or any combination thereof. The memory 230 contains an report annotation application 232. In numerous embodiments, the memory 230 further includes raw case reports 234 to be tested, and a base dictionary 236 containing a base set of standard words.

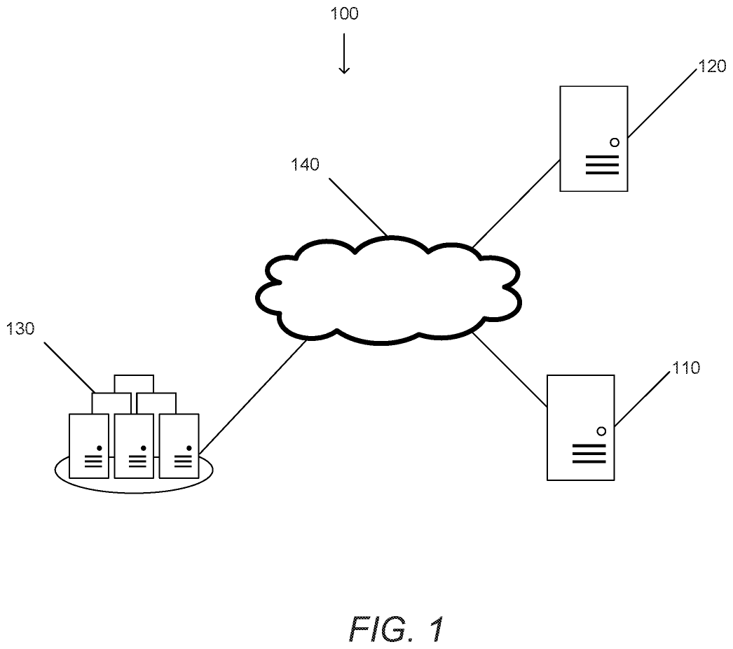

While a particular report annotation system and a particular report annotation server are illustrated in FIGS. 1 and 2, respectively, one of ordinary skill in the art can appreciate that any number of different architectures can be used as appropriate to the requirements of specific applications of embodiments of the invention without departing from the scope or spirit of the invention. Processes for report annotation are discussed in further detail below.

### Report Annotation Processes

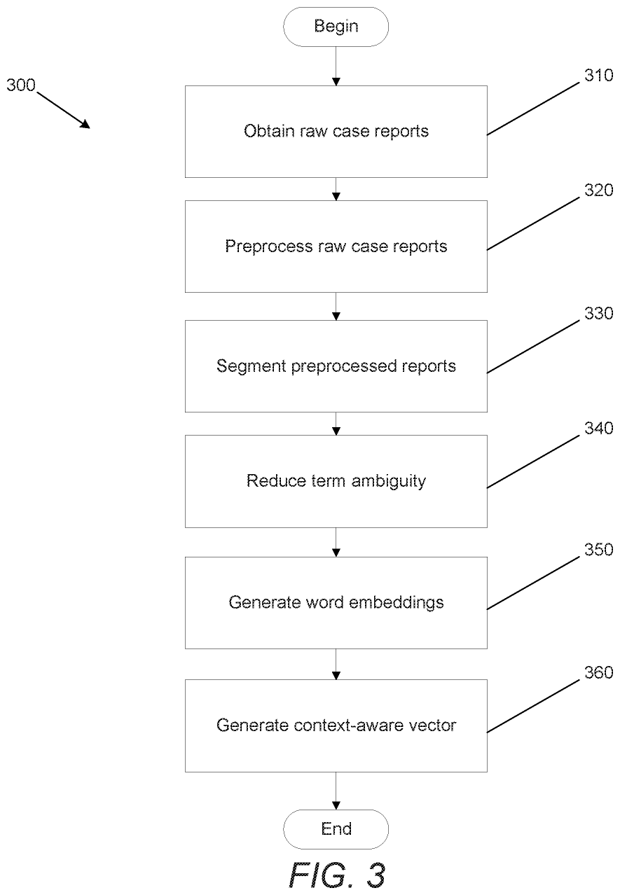

In many embodiments, report annotation processes involve annotating raw case reports from many different institutions with context-aware dense word embedding vectors using a two phase process. The first phase involves a semantic key term mapping, and the second phase involves a context analysis. Following pre-processing steps, semantic-dictionary mapping with domain specific key-terms are used as the basis of the word vector creation process. The semantic dictionary is also used to create a context-aware vector representation of whole reports based on windowing of the domain-specific key-terms. Finally, a supervised classification model is trained to learn the mapping between the report vectors of a training set and ground truth labels for predicting the annotation of test cases. However, the majority of the process can be implemented in an unsupervised manner. Turning now to FIG. 3, a process for evaluating AI systems in accordance with an embodiment of the invention is illustrated.

Process 300 includes obtaining (310) raw case reports. Raw case reports are “raw” in the sense that they are in the form produced by a radiologist with no or minimal processing. In numerous embodiments, raw case reports are heterogenous and are collected from different medical institutions. In many embodiments, raw case reports include at least a radiology image with natural language notes by a radiologist. In a variety of embodiments, raw case reports conform to the Annotation and Image Markup (AIM) standard.

The process 300 includes preprocessing (320) the raw case reports. In various embodiments, the textual context of the raw case reports is stemmed and converted into a standard case (e.g. lowercase). Stopwords, punctuation characters, low frequency (˜<50) words, and words with few characters (˜<2) can be removed, and numbers can be converted into strings. In many embodiments, in order to preserve local dependencies, bigram collocations of all possible word-pairs are calculated for the entire pre-processed corpus of raw case reports. In many embodiments, this process is based on Pointwise Mutual Information. Bigrams with less than ˜50 occurrences can be discarded and the top ˜1000 bigram collocations are concatenated as a single word to improve the accuracy of the word embeddings. However, any number of NLP preprocessing steps can be applied as appropriate to the requirements of specific applications of embodiments of the invention.

The process 300 further includes segmenting (330) the preprocessed case reports. Often, case report standards delineate several sections that should be present in a report. Case reports can be segmented by separating the different sections of the reports, for example, by using regular expressions and/or natural language processing techniques. In some embodiments, only relevant sections of a report are used.

The process 300 further includes reducing (340) term ambiguity using semantic dictionary mapping. In various embodiments, domain ontologies can be exploited to reduce term ambiguity and improve the semantic accuracy of the reports. In numerous embodiments, this is done by using a lexical scanner that accurately recognizes corpus terms which share a common root or stem with predefined terminology, which is mapped to controlled terms (“key-terms”). In many embodiments, the predefined terminology is domain specific and/or stored as a dictionary.

Domain ontologies can be created in any of a number of ways. In some embodiments, a Simple Protocol and RDF Query Language (SPARQL) query engine can remotely query a RadLex lexicon to find the key-terms provided by domain experts and programmatically extract a sub-tree from the RadLex lexicon. However, any number of different databases can be queried depending on the subject of the obtained case reports. Further, any number of different query engines can be similarly constructed to interface with a given lexicon. The query can perform pattern matching on the available graph of the RadLex terminology and construct a domain-specific dictionary. In some embodiments, the constructed dictionary is reviewed to remove redundancies.

More than one lexicon can be used to construct a dictionary. For example, a specific lexicon like those in RadLex can be merged with a general terminology, such as CLEVER, which is designed to detect broadly applicable clinical contexts and map them to root terms. Domain-specific key-terms and general terms can be merged to create a robust set of key-terms. These key-terms can be used to reduce variation in reports via mapping, and to help generate context-aware vector representations to support categorization.

Process 300 further incudes generating (350) word-embeddings. In many embodiments, preprocessed reports are used to create vector embeddings for words in an unsupervised manner using the word2vec model. The word2vec model adopts distributional semantics to learn dense vector representations of all words in the pre-processed corpus by analyzing their context. In other words, the vectors produced represent each word or phrase as a mathematical combination of the words and phrases surrounding it within a linear context window.

Given the above term ambiguity reduction, the size of the vocabulary has been reduced by mapping words in the corpus to key-terms, but also the probability of OOV word encounters has been reduced. Therefore, the application of word2vec is facilitated. In many embodiments, word2vec is trained using a skip-gram model. In order to simplify and reduce compute complexity, in many embodiments, vectors are only built for terms occurring more than 5 times in the corpus.

Process 300 further includes generating (360) context-aware vectors. In many embodiments, the key-terms are used to identify the window of relevant words for generating a context-aware vector representation of whole reports. Key-terms in each report can be identified, and if a match is found in a given report, the context for the report can be defined as the key-term and a small arbitrary number of surrounding key words. In numerous embodiments, the arbitrary number is based on the average sentence length of the text in the corpus. The context's vector can be computed as the average of its constituent word vectors, which can be averaged using word2vec.

The report vector can be computed using the following formulation:

$${V}_{report} = \frac{1}{‖ N ‖} {\sum }_{c ∈ keyterms} ( \frac{1}{‖ N ‖} {\sum }_{w ∈ context} {v}_{w} )$$

where Vᵣₑₚₒᵣₜ is the report vector, v_{w} refers to the vector of word w inferred from the word2vec mode, n is the context window size (i.e. the arbitrary number), and N is the number of key-terms in the report.

An advantage of the context-aware vector representation is that it can preserve relevant information about the findings in each report while having a low dimensionality. This can reduce complexity when attempting to classify the reports in either the training phase or operative phase of an AI model.

Although specific methods for AI evaluation are discussed above with respect to FIG. 3, many different methods can be implemented in accordance with many different embodiments of the invention. It is therefore to be understood that the present invention may be practiced in ways other than specifically described, without departing from the scope and spirit of the present invention. Thus, embodiments of the present invention should be considered in all respects as illustrative and not restrictive. Accordingly, the scope of the invention should be determined not by the embodiments illustrated, but by the appended claims and their equivalents.

What is claimed is:

1. A report annotation server, comprising:
a processor; and
a memory containing a report annotation application, where the report annotation application configures the processor to:
obtain a plurality of case reports from at least one medical database;
preprocess the plurality of case reports;
segment the preprocessed plurality of case reports;
reduce the term ambiguity of the segmented plurality of case reports;
generate word embeddings; and
generate a context-aware vector based on the word embeddings.

2. The report annotation server of claim 1, wherein the report annotation application further configures the processor to annotate case reports in the plurality of case reports based on the context-aware vectors.

3. The report annotation server of claim 1, wherein case reports in the plurality of case reports comprise radiology images.

4. The report annotation server of claim 3, wherein case reports in the plurality of case reports conform to the AIM file standard.

5. The report annotation server of claim 1, wherein the report annotation application further directs the processor to segment the preprocessed plurality of case reports based on report section.

6. The report annotation server of claim 1, wherein to reduce the term ambiguity, the report annotation application further directs the processor to:
generate a domain ontology; and
identify words in segmented plurality of case reports that map to key-terms in the domain ontology.

7. The report annotation server of claim 6, wherein the domain ontology is based on a query of the RadLex lexicon.

8. The report annotation server of claim 7, wherein the query of the RadLex lexicon is merged with a general terminology dictionary.

9. The report annotation server of claim 1, wherein to generate word embeddings, the report annotation application directs the processor to use a word2vec model.

10. The report annotations server of claim 1, wherein to generate context aware vectors, the report annotation directs the processor to identify a window of relevant words based on the location of an identified key-term.

11. A method for annotation reports comprising:
obtaining a plurality of case reports from at least one medical database;
preprocessing the plurality of case reports;
segmenting the preprocessed plurality of case reports;
reducing the term ambiguity of the segmented plurality of case reports;
generating word embeddings; and
generating a context-aware vector based on the word embeddings.

12. The method of claim 1, further comprising annotating case reports in the plurality of case reports based on the context-aware vectors.

13. The method of claim 1, wherein case reports in the plurality of case reports comprise radiology images.

14. The method of claim 13, wherein case reports in the plurality of case reports conform to the AIM file standard.

15. The method of claim 1, where segmenting the preprocessed plurality of case reports is based on report section.

16. The method of claim 1, wherein reducing term ambiguity comprises:
generating a domain ontology; and
identifying words in segmented plurality of case reports that map to key-terms in the domain ontology.

17. The method of claim 16, wherein the domain ontology is based on a query of the RadLex lexicon.

18. The method of claim 17, wherein the query of the RadLex lexicon is merged with a general terminology dictionary.

19. The method of claim 1, wherein generating word embeddings comprises using a word2vec model.

20. The method of claim 1, wherein generating context aware vectors comprises identifying a window of relevant words based on the location of an identified key-term.
